(19) United States
(12) Patent Application Publication
(10) Pub. No.: US 2005/0177137 A1
(43) Pub. Date: Aug. 11, 2005

(54) Administering device with temperature sensor

(76) Inventor: Urs Kipfer, Luetzelflueh (CH)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/067,601

(22) Filed: Feb. 25, 2005

Related U.S. Application Data
(63) Continuation of application No. PCT/EP03/09476, filed on Aug. 27, 2003.
(60) Provisional application No. 60/407,812, filed on Sep. 3, 2002.

(30) Foreign Application Priority Data
Aug. 31, 2002 (CH) 1488/02
Aug. 27, 2003 (WO) PCT/EP03/09476

(51) Int. Cl. A61K 9/22

(52) U.S. Cl. 604/890.1

(57) ABSTRACT

A device for administering a fluid product, including a container for the fluid product, an apparatus for dispensing the fluid product from the container, a metering apparatus for adjusting a product dose, and at least one temperature sensor disposed inside the container or near the fluid product. The present invention also encompasses a method for controlling the administration of a fluid product, wherein a metered quantity of a product dose, which is to be administered, is adjusted in accordance with the temperature of the fluid product and/or the temperature of the surroundings of the fluid product.

### CROSS-REFERENCE TO RELATED APPLICATION(S)

This application is a continuation of International Application No. PCT/EP03/09476, filed on Aug. 27, 2003, which claims priority to U.S. Provisional Application No. 60/407,812, filed on Sep. 3, 2002 and Swiss Application No. 1488/02, filed on Aug. 31, 2002, the contents of which are incorporated in their entirety herein by reference.

### BACKGROUND

The present invention relates to devices and methods for administering a fluid product and to devices and methods for controlling the administering of a fluid product. More particularly, the present invention relates to an infusion apparatus for administering insulin.

Such devices are, for example, used in medical or therapeutic applications, for repeatedly or continuously administering a product dosage comprising a particular dose or amount of a fluid product. The fluid product can, for example, be a liquid active solution such as insulin, growth hormones, etc. The administering devices can be formed as stationary apparatus or as portable apparatus which the user carries around, for example on the body.

An infusion pump which serves to deliver or infuse an active solution is, for example, known from EP-B-0 143 895. The infusion pump comprises a casing in which a container for the active solution, an administering means for administering the active solution from the container and a dosing means for setting a particular product dosage to be administered are accommodated. A stopper is provided in the container and is accommodated such that it can shift in an advancing direction towards an outlet in order to delivery the active solution within the container. The administering means comprises a driven member and a drive means. The driven member is connected to the stopper in the container and is driven by the drive means in order to advance the stopper within the container. The dosing means sets a dosage comprising a desired dosage amount of the active substance. The dosage is for example set by limiting the advancing path of the stopper within the container in accordance with the dosage amount. The drive means can be activated by hand or an electric motor can be provided which serves as the drive for the driven member. The driven member and/or stopper within the container can also be advanced by a rotational mechanism in which a rotation is converted into a translational movement for driving the stopper. The active solution and/or fluid product is administered such that when the administering means is activated, the stopper is shifted by a set path length towards a container outlet and the active substance can thus be dispensed for example into a flexible infusion tube and introduced into a patient via an access, for example in the form of a cannula.

Such administering devices are generally partially pre-programmed, i.e., administering the fluid product is controlled via a memory and control unit, using which the size of a dosage amount, the administering frequency or other operations or operational parameters can be set or determined. Furthermore, conventional administering devices can have various control and display means in order to enable the administering of the product to be monitored. For this purpose, measuring systems, for example, may be used for recording various body parameters of a patient, such as measuring the pulse rate, blood pressure, blood sugar level, body temperature, etc. These parameters can then be relayed to the memory and control unit, in order to control the administering. This can, for example, improve the delivery accuracy of the fluid product or detect occlusions or leaks within a conduit system for the fluid product.

Up until now, however, variable parameters of the fluid product have not been considered when controlling such administering devices. The delivery accuracy is for example affected by temperature fluctuations to which the administering device and therefore the fluid product are subjected. The fluid product can exhibit different forms of state depending on temperature. A solvent in which an active substance is dissolved can for example exhibit a greater volume at higher temperatures, such that the concentration of the active substance in the solution changes. Furthermore, the active substance itself can change its properties in accordance with a particular temperature. A change in the fluid product with temperature also changes the amount of the active substance which is dispensed when a set product dosage is administered. In this way, a patient can be given too much or too little active substance.

Measurements have for example shown that for insulin in a container of for example 300 units, temperature fluctuations with temperature differences of 15° C. can result in an inaccuracy of ±1 unit. Such a temperature fluctuation can, for example, occur when going to bed or spending a short time in the sun. Many diabetics have an average insulin delivery rate of for example 0.5 units per hour. A temperature-dependent inaccuracy in the administered dosage amount can therefore easily result in the insulin being incorrectly dosed and the patient therefore being incorrectly treated.

### SUMMARY

It is an object of the present invention to provide a device for administering a fluid product and a method for controlling the administering of the fluid product, wherein the dosage setting and dispensing accuracy of a product dosage are improved, wherein temperature fluctuations are taken into account and it is ensured that the administering device functions reliably.

The object is addressed by a device in accordance with the present invention, the device comprising a container for the fluid product, a dispensing means for administering the fluid product from the container, a dosing means for setting a product dosage, and at least one temperature sensor for measuring the temperature of the fluid product and/or the temperature in the vicinity of the fluid product.

The object is also addressed by a method in accordance with the present invention, the method involving the delivery of a fluid product such as a medicinal product from a container, the method comprising the steps of providing a fluid product to be delivered, setting a dosage amount of the fluid product to be delivered or administered by using a dosing means, wherein the dosage amount is set in accordance with the temperature of the fluid product and/or in accordance with the temperature of the environment near the container.

Accordingly, a device for administering a fluid product, for instance a liquid active solution, comprises a container for the fluid product, a dispensing means for administering the fluid product from the container and a dosing means for setting a product dosage. The product container can for example be provided by a conventional ampoule, or the fluid product can be poured into a chamber of the administering device which serves as the container. As described above, the container comprises an outlet for dispensing the product from the container and a stopper which can be moved in the advancing direction by the dispensing means. In some embodiments, the dosing means preferably comprises a rotational mechanism in which, in order to set a product dosage by setting a particular rotational position of a rotational member, the advancing path of the stopper within the container is limited. A drainage conduit for dispensing the fluid product is connected to the container outlet. For this purpose, a flexible tube can be provided and a cannula which is arranged on a surface of a body tissue of the patient and leads into said tissue.

In accordance with the invention, the administering device comprises at least one temperature sensor which measures the temperature of the fluid product in the container and/or the temperature in the vicinity of the fluid product and/or the container. For this purpose, in some embodiments, the at least one temperature sensor is arranged on or in the container. A number of temperature sensors can also be provided, which may be provided at different, selected locations on or in the container and/or the administering device, in order to enable the temperature to be measured accurately by comparing temperatures.

In order to control the administering of the fluid product from the administering device described above, in some embodiments, the administering device comprises a control unit comprising a memory and a battery. In some embodiments, the control unit can be used, with or without the aid of a dosing means, to set the amount or dose of product to be administered. In the latter case, the control unit may comprise the dosing means. In accordance with the invention, the dosage amount is set in accordance with the temperature of the fluid product and/or in accordance with the temperature of the environment near the container or near the administering device. For an administering control method in accordance with the invention, a temperature value measured by a temperature sensor can, for example, be output or communicated to the control unit, whereupon the control unit determines the setting of the dosage amount by means of the dosing means and correspondingly controls the dosing means and/or corrects the setting.

Determining the temperature of the fluid product or the temperature in the vicinity of the fluid product and taking into account this temperature when setting the dosage can significantly improve the dispensing accuracy of an active substance to a patient. Under- or overdosing can be prevented. This enables the success of a therapy or the well-being of a patient to be significantly increased.

### BRIEF DESCRIPTION OF THE DRAWINGS

FIG. 1 depicts, in schematic form, an administering device in accordance with the present invention.

### DETAILED DESCRIPTION

One preferred embodiment of the present invention is formed by an administering device in the form of a portable insulin pump, for example an insulin pump of the type that is well-known to those skilled in art, in which at least one temperature sensor is arranged within a chamber in the insulin pump into which an ampoule can be inserted, Typically, the ampoule is filled with the fluid product. Advantageously, the temperature sensor is directly adjacent to the ampoule. It would also be possible to form the chamber such that the fluid product can be poured directly into the chamber; in this case, the temperature sensor comes into direct contact with the fluid product. Another temperature sensor can be arranged on the outside of the administering device, e.g., on a casing or casing portion of the device. Conventional sensors, which are available cost-effectively as mass-produced products, can be used as temperature sensors.

The administering device in accordance with the invention preferably comprises an alarm means which outputs an alarm signal when a predetermined temperature and/or predetermined temperature difference with respect to a settable reference temperature is exceeded. The alarm signal can be provided by a suitable signal, such as an acoustic, visual and/or vibration signal. If a temperature signal is transmitted from the temperature sensor to the control unit which exceeds a predetermined temperature and/or predetermined temperature difference, the dosing means can be automatically controlled to adjust a product dosage, or the user can decide on the basis of the alarm signal whether he wishes to change the dosage or not.

In one preferred embodiment, the administering device comprises a monitoring means for monitoring an occlusion or a leak within the device, which comprises sensors within the administering device, for monitoring. Such occlusions and/or leak sensors are based on measuring a force or pressure acting on the fluid product or the dispensing means, and/or measuring the force necessary from the dispensing means for administering. The readings from the sensors are transmitted to the control unit which, when a limit value of the readings is exceeded, interrupts or prevents the fluid product from being administered by the administering device, and/or outputs an alarm signal. The readings from such sensors are, however, dependent on the temperature of the fluid product, i.e., the force to be expended for administering or the pressure prevalent in the container fluctuate with temperature. In accordance with the invention, the limit values used by the monitoring means for monitoring an occlusion or a leak are set or corrected in accordance with the temperature of the fluid product and/or in accordance with the temperature of the environment near the product container.

In the administering control method in accordance with the present invention, the dosage amount of a product dosage is preferably set in accordance with a temperature difference with respect to a reference temperature. Room temperature or the temperature prevalent on a patient's body at the location at which an administering apparatus is worn can, for example, be used as the reference temperature. In the event of a particular deviation from this reference value, i.e., when a particular temperature difference is exceeded, a product dosage setting which is correct for the reference temperature can then be adjusted and/or corrected in accordance with the level of the temperature difference. This operational control enables and/or enhances dispensing the product amount required for a successful treatment of the patient. Accordingly, the predetermined dosage amount is changed such that the desired amount is also actually dispensed. When a temperature difference is exceeded, an alarm signal can be outputted by the alarm means, such that the user can independently perform or trigger a correction. However, the dosage is advantageously corrected automatically by the control unit. The dosage setting and the correction can then be indicated on a display. The correction, however, need not necessarily be displayed, since due to the correction, the desired dosage amount indicated on the display is maintained.

In some preferred embodiments, the dosage amount is corrected when the temperature difference is exceeded within a predetermined period of time. In the case of merely brief fluctuations in temperature, when the pre-set temperature difference and/or reference temperature is therefore only briefly exceeded, it may be that a correction is not necessary. For example, the state of the fluid product within the container only changes once an increased or reduced temperature acts on the product over a certain period of time. It is therefore useful to take into account the change in temperature over time when controlling the administering of product.

The idea of the invention has been illustrated by way of an administering device having an open system, i.e., a fluid is dispensed from the device. In principle, however, it is also conceivable to employ the features of the present invention in a closed system, for example in a measuring method having a closed fluid cycle, in which a measuring liquid is used as the fluid.

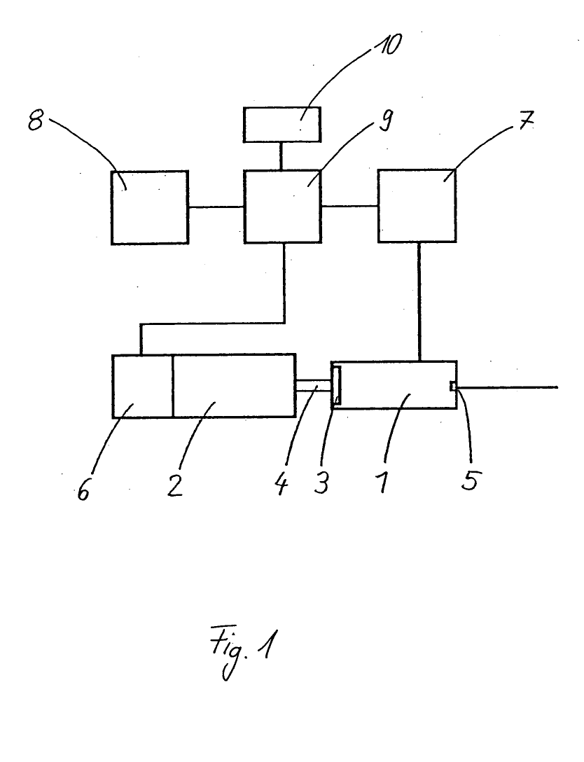

Embodiments of the device and method of the present invention are further explained by way of the exemplary embodiment of a device shown in FIG. 1.

A device for administering a fluid product, such as is shown in the drawing, comprises a container 1 for the fluid product and a dispensing means 2 for administering the fluid product from the container 1. The container 1 is formed such that it can accommodate an ampoule containing the fluid product. For this purpose, a stopper 3 is arranged in the container 1 and is advanced towards an outlet 5 of the container 1 by a driven member 4 of the dispensing means 2. The administering device further comprises a dosing means 6 for setting a dosage amount of a product dosage of the fluid product. A temperature sensor 7 is arranged on an inner wall of the container 1 and comes to rest next to the ampoule when the ampoule is introduced. The administering device further comprises a monitoring means 8 comprising an occlusion and leak sensor for monitoring an occlusion and/or leak within the administering device.

The temperature sensor 7, the occlusion and leak sensor, the dosing means 6 and the dispensing means 2 are connected to a control unit 9. The control unit 9 comprises at least one memory, for example for storing particular administering or patient parameters.

The temperature value measured by the temperature sensor 7 in the container 1, i.e., near the fluid product, is outputted or sent to the control unit 9. In accordance with the temperature measured by the sensor 7, the control unit 9 corrects a dosage setting on the dosing means 6 and/or the administering of the fluid product by the dispensing means 2. That is to say, the advancing path travelled by the stopper 3 for administering is changed in accordance with the measured temperature. Furthermore, the limit values of the monitoring means 8, used to monitor an occlusion or leak, are adjusted in accordance with the temperature measured by the temperature sensor 7.

A display 10 serves to display various parameters of the administering device, such as a dosage setting, and can indicate a visual warning signal when a temperature difference measured by the temperature sensor 7 is exceeded. When a warning is indicated, the user can correct a dosage via operating buttons.

In the foregoing description, embodiments of the present invention have been presented for the purpose of illustration and description. They are not intended to be exhaustive or to limit the invention to the precise forms or steps disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A device for administering a fluid product, comprising:
a) a container for the fluid product;
b) a dispensing means for administering the fluid product from the container;
c) a dosing means for setting a product dosage; and
d) at least one temperature sensor for measuring at least one of the temperature of the fluid product and the temperature in the vicinity of the fluid product.

2. The administering device as set forth in claim 1, wherein the at least one temperature sensor is arranged on or in the container.

3. The administering device as set forth in claim 1, further comprising alarm means for outputting an alarm signal when a temperature difference with respect to a reference temperature is exceeded.

4. The administering device as set forth in claim 3, wherein the alarm signal is an acoustic, visual and/or vibration alarm signal.

5. The administering device as set forth in claim 1, wherein the dosing means sets the product dosage in accordance with the temperature measured by the temperature sensor.

6. The administering device as set forth in claim 1, further comprising monitoring means for monitoring an occlusion and/or leak in the administering device, said monitoring means settable in accordance with the temperature measured by the temperature sensor.

7. A method for controlling the administering of a fluid product from a container for the fluid product, comprising using a dosing means to set a dosage amount of a product dosage to be administered, wherein the dosage amount is set in accordance with at least one of a temperature of the fluid product and a temperature of the environment near the container.

8. The method as set forth in claim 7, wherein the dosage amount is set in accordance with a temperature difference with respect to a reference temperature.

9. The method as set forth in claim 8, wherein a predetermined dosage amount is changed when the temperature difference is exceeded.

10. The method as set forth in any one of claim 9, wherein the predetermined dosage amount is changed when the temperature difference is exceeded within a predetermined period of time.

11. The method as set forth in any one of claim 7, wherein the dosage amount is changed automatically.

12. The method as set forth in any one of claim 10, wherein the dosage amount is changed automatically.

13. The method as set forth in any one of claims 8, wherein an alarm is outputted when the temperature difference is exceeded.

14. The method as set forth in claim 7, further comprising providing a monitoring means for monitoring an occlusion and/or leak in an administering device, wherein a limit value of the monitoring means is set in accordance with at least one of the temperature of the fluid product and the temperature of the environment near the container.
